(19) United States
(12) Patent Application Publication
(10) Pub. No.: US 2023/0289959 A1
(43) Pub. Date: Sep. 14, 2023

(54) THREE-DIMENSIONAL OBJECT, TRAINING SYSTEM, THREE-DIMENSIONAL OBJECT PRODUCING METHOD, AND METHOD FOR EVALUATING ACCURACY OF THREE-DIMENSIONAL OBJECT

(71) Applicants: Tatsuya Niimi, Kanagawa (JP); Takashi Matsumura, Kanagawa (JP); Takuya Saito, Kanagawa (JP)

(72) Inventors: Tatsuya Niimi, Kanagawa (JP); Takashi Matsumura, Kanagawa (JP); Takuya Saito, Kanagawa (JP)

(73) Assignee: Ricoh Company, Ltd., Tokyo (JP)

(21) Appl. No.: 18/040,008

(22) PCT Filed: Jul. 30, 2021
(86) PCT No.: PCT/JP2021/028415

§ 371 Date: Jan. 31, 2023

(30) Foreign Application Priority Data
Aug. 7, 2020 (JP) 2020-135170
Aug. 7, 2020 (JP) 2020-135171
Aug. 7, 2020 (JP) 2020-135175

(51) Int. Cl. G06T 7/00; G16H 30/40

(52) U.S. Cl. CPC G06T 7/0012; G16H 30/40; G06T 2207/10088

(57) ABSTRACT

A three-dimensional object can be produced based on biological property information indicating a biological property obtained by MRE measurement of an organism. The three-dimensional object has a distribution of a strength property corresponding to a distribution of the biological property.

### TECHNICAL FIELD

The present disclosure relates to a three-dimensional object, a training system, a three-dimensional object producing method, and a method for evaluating accuracy of a three-dimensional object.

### BACKGROUND ART

Three-dimensional object producing techniques have made a remarkable progress and various 3D printers of a fused deposition modeling type, a binder jetting type, a stereolithography type, a powder sintering additive manufacturing type, and a material jetting type have been used in the manufacturing industry. As the materials used in the 3D printers, various materials have been being developed, including metals and resins that have been hitherto known. New uses suited to these various materials have also been proposed, and applications of 3D printers to, for example, the medical field and the healthcare field have been being expected, as well as to the industrial field. Examples of applications of 3D printers to the medical field include production of implantable artificial bones using, for example, titanium, hydroxyapatite, and PEEK, and studies into artificial organs obtained by direct lamination of cells in layers. Examples of applications of 3D printers to the healthcare field include applications to, for example, hearing aids and artificial limbs that need to reflect individual-specific shapes. What is behind this expanded ranges of applications is easy availability of 3D data by use of, for example, 3D scanners, CT, and MRI.

Other examples of expected applications of 3D printers to the medical field include applications to models that imitate actual organ shapes and organisms for surgical trainings and simulations. Within the background context of the expectation for applications to these models, there are the recent progress in the development of medical devices and the accompanying ongoing shift in the medical trend from the existing medicine including major incisions and excisions to low-invasive, low patient-burdening medicine by, for example, catheters, endoscopes, and robotic assists, and the need for very high-level techniques and skills in the medical operations including use of the mentioned medical devices and the accompanying recognition of the importance of surgical trainings using suitable models for preventing medical accidents. Moreover, for difficult surgeries with few actual cases done, if models that reproduce the details of the target sites can be obtained beforehand, it is possible to perform scrupulous preoperative simulations.

A disclosed method irradiates a photo-curable resin with laser light based on two-dimensional tomographic image data captured by a tomographic shape measuring device such as a CT device to form a cured layer matching each tomographic shape, and laminates such cured layers sequentially to produce a three-dimensional model imitating, for example, an organ (for example, see PTL 1).

Another disclosed method generates blueprint data that indicates two or more of organ shape, organ hardness, organ density, and effective atomic number on an approximately individual voxel-by-voxel basis based on a medical 3D image including a plurality of voxels that represent the organ of the subject, and produces a 3D object based on the blueprint data (for example, see PTL 2).

Examples of known models that imitate, for example, organs include models produced by 3D printers and formed of hard materials such as acrylic-based resins and urethane-based resins. However, hard materials are hard as materials, and cannot easily reproduce the texture of actual organs.

Examples of other known models that imitate, for example, organs include models produced by a casting method using a template and formed of water-based gels such as silicone elastomers and polyvinyl alcohols (for example, see PTL 3). In production by the casting method, there is a wide selection range of materials, and it is possible to produce models using materials close to the texture of actual organs, such as polyvinyl alcohols. However, the casting method produces a model in an integrated shape using a single material. Therefore, the casting method cannot reproduce a distribution of a property in an organ, attributable to the site of an organ to be produced or attributable to the area of disease.

Moreover, it is impossible to directly touch actual organs and measure the textures of the organs. Therefore, there is generally a need to adapt models to textures close to the actual organs based on the result of hearing from doctors. However, each patient about whom the doctors are asked in the hearing has their own different texture data. Hence, it is difficult to produce a model that reproduces the texture of an organ of a specific patient.

### CITATION LIST

### Patent Literature

PTL 1: Japanese Unexamined Patent Application Publication No. 05-11689
PTL 2: Japanese Unexamined Patent Application Publication No. 2019-153180
PTL 3: Japanese Unexamined Patent Application Publication No. 2015-069054

### SUMMARY OF INVENTION

### Technical Problem

The present disclosure has an object to provide a three-dimensional object that accurately reproduces the distribution of a property in an organism, and a three-dimensional object producing method for producing a three-dimensional object that accurately reproduces the distribution of a property in an organism.

### Solution to Problem

The present disclosure relates to a three-dimensional object produced based on biological property information indicating a biological property obtained by MRE measurement of an organism. The three-dimensional object has a distribution of a strength property corresponding to a distribution of the biological property.

The present disclosure relates to an object producing method including an object producing step of producing a three-dimensional object using a 3D printer based on medical 3D data of an organism. The medical 3D data includes biological property information indicating a biological property obtained by MRE measurement of the organism. The three-dimensional object has a distribution of a strength property corresponding to a distribution of the biological property.

### Advantageous Effects of Invention

The present disclosure can provide a three-dimensional object that accurately reproduces the distribution of a property in an organism, and a three-dimensional object producing method for producing a three-dimensional object that accurately reproduces the distribution of a property in an organism.

### BRIEF DESCRIPTION OF DRAWINGS

FIG. 1 is an exemplary view illustrating an example of a medical image data set.

FIG. 2 is an exemplary view illustrating an example of medical 3D data.

FIG. 3 is an exemplary view illustrating an example of medical 3D data.

FIG. 4 is an exemplary diagram illustrating an example of region-specific medical 3D data obtained by dividing medical 3D data on a voxel region basis.

FIG. 5 is an exemplary diagram illustrating an example of STL-format data obtained by converting region-specific medical 3D data to a surface model.

FIG. 6 is an exemplary diagram illustrating an example of FAV-format data obtained by converting region-specific medical 3D data to a FAV format.

FIG. 7 is an exemplary diagram illustrating an example of a seven-gradation strength property expressed by arrangement patterns of a high-strength object producing composition and a low-strength object producing composition.

FIG. 8 is an exemplary view illustrating an example of a water-swellable layered clay mineral serving as a mineral, and a dispersed state of the water-swellable layered clay mineral in water.

FIG. 9 is an exemplary view illustrating an example of a three-dimensional object producing apparatus.

FIG. 10 is an exemplary view illustrating an example of a three-dimensional object detached from supports.

FIG. 11 is a graph plotting measurements of compressive strength of samples 1 to 4.

FIG. 12 is a graph plotting measurements obtained in a tensile test of samples 5 to 8.

### DESCRIPTION OF EMBODIMENTS

1. Three-Dimensional Object

In the present disclosure, a “three-dimensional object” (also referred to as model) means an object produced to have a three-dimensional shape and imitate a real object. Examples of the three-dimensional object include an organ model imitating the shape of at least part of an organ and properties of the organ. Organs are functional organs constituting an organism, and encompass not only internal organs but also all kinds of organs that constitute an organism, such as bones, skin, and blood vessels.

The material constituting an organ model is not particularly limited so long as the material can produce a three-dimensional object imitating the shape of at least part of an organ and the properties of the organ. For example, a material satisfying the following three conditions is preferable.
A material that is soft, and of which properties can be controlled
A self-standing material (that can retain a shape)
A material that can be formed into an object using an object producing apparatus (also referred to as “3D printer”)

Examples of such materials include soft plastics containing, for example, polyurethane resins and silicone resins, and hydrogels containing, for example, polyvinyl alcohols. Hydrogels are preferable. In the present disclosure, a “hydrogel” means a structural body formed by water being embraced in a three-dimensional network structure containing a polymer. When such a three-dimensional network structure is a three-dimensional network structure formed by a polymer being combined with a mineral, the three-dimensional network structure is referred to particularly as “organic-inorganic-combined hydrogel”. A hydrogel contains water as a main component. Specifically, a hydrogel contains water preferably by 30.0% by mass or greater, more preferably by 40.0% by mass or greater, and yet more preferably by 50.0% by mass or greater relative to the total amount of the hydrogel.

The material constituting an organ model is formed of an object producing composition, which is the precursor of the material. Here, when the material constituting an organ model is a hydrogel, the hydrogel is formed of a three-dimensional hydrogel object producing composition, which is the precursor of the hydrogel. In the present disclosure, a “three-dimensional hydrogel object producing composition” means a liquid composition that cures and forms a hydrogel in response to irradiation with active energy rays such as light or irradiation with heat, and is particularly used for producing a three-dimensional object formed of a hydrogel. The three-dimensional hydrogel object producing composition contains water and a polymerizable compound, and may contain a mineral, an organic solvent, and other components as needed. Each component contained in the three-dimensional hydrogel object producing composition will be described below.

(1) Water

The three-dimensional hydrogel object producing composition contains water. The water is not particularly limited, and any water that is ordinarily used as solvents may be used. Examples of the water include pure water such as ion-exchanged water, ultra-filtrated water, reverse osmotic water, and distilled water, and ultrapure water.

When the three-dimensional hydrogel object producing composition is used for producing an organ model, the content of the water is preferably 30.0% by mass or greater but 90.0% by mass or less and more preferably 40.0% by mass or greater but 90.0% by mass or less relative to the total amount of the three-dimensional hydrogel object producing composition.

Any other component such as an organic solvent may be dissolved or dispersed in the water in order to, for example, impart a moisture retaining property, an antibacterial activity, and conductivity, and adjust hardness.

(2) Polymerizable Compound

The three-dimensional hydrogel object producing composition contains a polymerizable compound, which is a compound containing a polymerizable functional group. Examples of the polymerizable compound include monomers and oligomers. The polymerizable compound polymerizes and forms at least part of a polymer in response to irradiation with active energy rays or heat. That is, a polymer contains a structural unit attributable to the polymerizable compound. It is also preferable that a polymer be crosslinked and combined with a mineral to form a three-dimensional network structure in a hydrogel. It is preferable that the polymerizable compound be water-soluble. For example, water solubility means a solubility of a monomer by 90% by mass or greater thereof when the monomer (1 g) is mixed and stirred in water (100 g) at 30 degrees C.

The polymerizable compound is not particularly limited so long as the polymerizable compound is a compound containing a polymerizable functional group. A compound containing a photopolymerizable functional group is preferable. In the present disclosure, a “polymerizable functional group” means a functional group that undergoes a polymerization reaction in response to irradiation with active energy rays or application of heat, and a “photopolymerizable functional group” particularly means a functional group that undergoes a polymerization reaction in response to irradiation with active energy rays. The photopolymerizable functional group is not limited to as described above, and examples of the photopolymerizable functional group include: groups containing an ethylenic unsaturated bond, such as a (meth)acryloyl group, a vinyl group, and an allyl group; and cyclic ether groups such as an epoxy group. Specific examples of the compound that contains a group containing an ethylenic unsaturated bond include compounds containing a (meth)acrylamide group, (meth)acrylate compounds, compounds containing a (meth)acryloyl group, compounds containing a vinyl group, and compounds containing an allyl group.

(I) Monomer

A monomer that can be used as the polymerizable compound is a compound that contains one or more polymerizable functional groups, a preferable example of which is an unsaturated carbon-carbon bond. Examples of the monomer include monofunctional monomers and multifunctional monomers. Examples of the multifunctional monomers include bifunctional monomers and trifunctional or higher monomers. One of these monomers may be used alone or two or more of these monomers may be used in combination.

(A) Monofunctional Monomer

Examples of the monofunctional monomer include acrylamide, N-substituted acrylamide derivatives, N,N-disubstituted acrylamide derivatives, N-substituted methacrylamide derivatives, N,N-disubstituted methacrylamide derivatives, acrylic acid, 2-ethylhexyl (meth)acrylate, 2-hydroxyethyl (meth)acrylate, 2-hydroxypropyl (meth)acrylate, caprolactone-modified tetrahydrofurfuryl (meth)acrylate, 3-methoxybutyl (meth)acrylate, tetrahydrofurfuryl (meth)acrylate, lauryl (meth)acrylate, 2-phenoxyethyl (meth)acrylate, isodecyl (meth)acrylate, isooctyl (meth)acrylate, tridecyl (meth)acrylate, caprolactone (meth)acrylate, and ethoxylated nonylphenol (meth)acrylate. One of these monofunctional monomers may be used alone or two or more of these monofunctional monomers may be used in combination. Among these monofunctional monomers, acrylamide, N,N-dimethyl acrylamide, Nisopropyl acrylamide, acryloylmorpholine, hydroxyethyl acrylamide, and isobornyl (meth)acrylate are preferable.

The content of the monofunctional monomer is preferably 0.5% by mass or greater but 30.0% by mass or less relative to the total amount of the three-dimensional hydrogel object producing composition.

(B) Bifunctional Monomer

Examples of the bifunctional monomer include tripropylene glycol di(meth)acrylate, triethylene glycol di(meth)acrylate, tetraethylene glycol di(meth)acrylate, polypropylene glycol di(meth)acrylate, neopentyl glycol hydroxypivalic acid ester di(meth)acrylate, hydroxypivalic acid neopentyl glycol ester di(meth)acrylate, 1,3-butanediol di(meth)acrylate, 1,4-butanediol di(meth)acrylate, 1,6-hexanediol di(meth)acrylate, 1,9-nonanediol di(meth)acrylate, diethylene glycol di(meth)acrylate, neopentyl glycol di(meth)acrylate, tripropylene glycol di(meth)acrylate, caprolactone-modified hydroxypivalic acid neopentyl glycol ester di(meth)acrylate, propoxylated neopentyl glycol di(meth)acrylate, ethoxy-modified bisphenol A di(meth)acrylate, polyethylene glycol 200 di(meth)acrylate, and polyethylene glycol 400 di(meth)acrylate. One of these bifunctional monomers may be used alone or two or more of these bifunctional monomers may be used in combination.

(C) Trifunctional or Higher Monomer

Examples of the trifunctional or higher monomer include trimethylolpropane tri(meth)acrylate, pentaerythritol tri(meth)acrylate, dipentaerythritol hexa(meth)acrylate, triallyl isocyanurate, ε-caprolactone-modified dipentaerythritol tri(meth)acrylate, ε-caprolactone-modified dipentaerythritol tetra(meth)acrylate, ε-caprolactone-modified dipentaerythritol penta(meth)acrylate, ε-caprolactone-modified dipentaerythritol hexa(meth)acrylate, tris(2-hydroxyethyl)isocyanurate tri(meth)acrylate, ethoxylated trimethylolpropane tri(meth)acrylate, propoxylated trimethylolpropane tri(meth)acrylate, propoxylated glyceryl tri(meth)acrylate, pentaerythritol tetra(meth)acrylate, ditrimethylolpropane tetra(meth)acrylate, dipentaerythritol hydroxypenta (meth)acrylate, ethoxylated pentaerythritol tetra(meth)acrylate, and penta(meth)acrylate ester. One of these trifunctional or higher monomers may be used alone or two or more of these trifunctional or higher monomers may be used in combination.

The content of the multifunctional monomer is preferably 0.01% by mass or greater but 10.0% by mass or less relative to the total amount of the three-dimensional hydrogel object producing composition.

(II) Oligomer

An oligomer is a low polymer of the monofunctional monomer described above or a low polymer that contains a reactive unsaturated bond group at an end thereof. One of such oligomers may be used alone or two or more of such oligomers may be used in combination.

(3) Mineral

It is preferable that the three-dimensional hydrogel object producing composition contain a mineral. The mineral is not particularly limited and may be appropriately selected depending on the intended purpose so long as the mineral can bond with a polymer formed of the polymerizable compound described above. Examples of the mineral include layered clay minerals, and water-swellable layered clay minerals in particular.

A water-swellable layered clay mineral will be described with reference to FIG. 8. FIG. 8 is an exemplary view illustrating an example of a water-swellable layered clay mineral serving as the mineral, and a dispersed state of the water-swellable layered clay mineral in water. As illustrated in the upper section of FIG. 8, a water-swellable layered clay mineral is present in a state of single layers, and has a multilayered state of two-dimensional disk-shaped crystals having a unit lattice in the crystals. When the water-swellable layered clay mineral in the upper section of FIG. 8 is dispersed in water, the respective single layers separate into a plurality of two-dimensional disk-shaped crystals as illustrated in the lower section of FIG. 8.

Water swellability means a property of a layered clay mineral being dispersed in water with water molecules inserted between the respective single layers thereof as illustrated in FIG. 8. The shape of the single layers of the water-swellable layered clay mineral is not limited to the disk shape. The single layers of the water-swellable layered clay mineral may have any other shape.

Examples of the water-swellable layered clay mineral include water-swellable smectite, and water-swellable mica. More specific examples of the water-swellable layered clay mineral include water-swellable hectorite containing sodium as an interlayer ion, water-swellable montmorillonite, water-swellable saponite, and water-swellable synthetic mica. One of these water-swellable layered clay minerals may be used alone or two or more of these water-swellable layered clay minerals may be used in combination. Among these water-swellable layered clay minerals, water-swellable hectorite is preferable because a highly elastic hydrogel can be obtained.

The water-swellable hectorite may be an appropriately synthesized product or a commercially available product. Examples of the commercially available product include synthetic hectorite (LAPONITE XLG, available from Rock Wood), SWN (available from Coop Chemical Ltd.), and fluorinated hectorite SWF (available from Coop Chemical Ltd.). Among these commercially available products, synthetic hectorite is preferable in terms of improving the elastic modulus of a hydrogel.

The content of the mineral is preferably 1.0% by mass or greater but 40.0% by mass or less relative to the total amount of the three-dimensional hydrogel object producing composition.

(4) Organic Solvent

The three-dimensional hydrogel object producing composition may contain an organic solvent as needed. The organic solvent is contained in order to, for example, improve the moisture retaining property of a hydrogel.

Examples of the organic solvent include alkyl alcohols containing from one through four carbon atoms, amides, ketones, ketone alcohols, ethers, polyvalent alcohols, polyalkylene glycols, lower alcohol ethers of polyvalent alcohols, alkanolamines, and N-methyl-2-pyrrolidone. One of these organic solvents may be used alone or two or more of these organic solvents may be used in combination.

Among these organic solvents, polyvalent alcohols are preferable in terms of a moisture retaining property. Specifically, polyvalent alcohols such as ethylene glycol, propylene glycol, 1,2-propanediol, 1,2-butanediol, 1,3-butanediol, 1,4-butanediol, diethylene glycol, triethylene glycol, 1,2,6-hexanetriol, thioglycol, hexylene glycol, and glycerin can be suitably used.

The content of the organic solvent is preferably 10.0% by mass or greater but 50.0% by mass or less relative to the total amount of the three-dimensional hydrogel object producing composition. When the content of the organic solvent is 10.0% by mass or greater, drying of the three-dimensional hydrogel object producing composition can be suppressed. When the content of the organic solvent is 50.0% by mass or less, dispersibility of the mineral in the three-dimensional hydrogel object producing composition can be improved.

(5) Other Components

The three-dimensional hydrogel object producing composition may contain other components as needed.

The other components are not particularly limited and may be appropriately selected depending on the intended purpose. Examples of the other components include a stabilizer, a surface treating agent, a polymerization initiator, a colorant, a viscosity modifier, a tackifier, an antioxidant, an age resister, a cross-linking agent, an ultraviolet absorbent, a plasticizer, a preservative, metal ions, a filler, metal particles, and a dispersant.

(I) Stabilizer

A stabilizer is contained in order to disperse the mineral stably and maintain the three-dimensional hydrogel object producing composition in a sol state. When the three-dimensional hydrogel object producing composition is used in a system configured to discharge the three-dimensional hydrogel object producing composition in the form of a liquid droplet, a stabilizer is contained in order to stabilize the properties of the three-dimensional hydrogel object producing composition as a liquid.

Examples of the stabilizer include high-concentration phosphates, glycols, and nonionic surfactants.

(II) Surface Treating Agent

Examples of the surface treating agent include polyester resins, polyvinyl acetate resins, silicone resins, coumarone resins, fatty acid esters, glycerides, and waxes.

(III) Polymerization Initiator

Examples of the polymerization initiator include thermal polymerization initiators and photopolymerization initiators. Of these polymerization initiators, photopolymerization initiators that produce radicals or cations in response to irradiation with active energy rays are preferable in terms of storage stability.

As a photopolymerization initiator, an arbitrary substance that produces radicals in response to irradiation with light (particularly, ultraviolet rays having a wavelength of 220 nm or longer but 400 nm or shorter) can be used.

The thermal polymerization initiator is not particularly limited and may be appropriately selected depending on the intended purpose. Examples of the thermal polymerization initiator include azo-based initiators, peroxide initiators, persulfate initiators, and redox (oxido-reduction) initiators.

(6) Properties of Three-Dimensional Hydrogel Object Producing Composition

The viscosity of the three-dimensional hydrogel object producing composition at 25 degrees C. is preferably 3.0 mPa s or higher but 20.0 mPa s or lower and more preferably 6.0 mPa s or higher but 12.0 mPa s or lower. When the viscosity of the three-dimensional hydrogel object producing composition is 3.0 mPa s or higher but 20.0 mPa s or lower, the three-dimensional hydrogel object producing composition can be suitably applied to liquid droplet discharging by 3D printers (particularly, of a material jetting type). The viscosity can be measured with, for example, a rotary viscometer (VISCOMATE VM-150III, available from Toki Sangyo Co., Ltd.).

The surface tension of the three-dimensional hydrogel object producing composition is preferably 20 mN/m or higher but 45 mN/m or lower and more preferably 25 mN/m or higher but 34 mN/m or lower. When the surface tension of the three-dimensional hydrogel object producing composition is 20 mN/m or higher, discharging stability of the three-dimensional hydrogel object producing composition can be improved. When the surface tension of the three-dimensional hydrogel object producing composition is 45 mN/m or lower, the three-dimensional hydrogel object producing composition can be easily filled into, for example, discharging nozzles for object production. The surface tension can be measured with, for example, a surface tensiometer (AUTOMATIC CONTACT ANGLE METER DM-701, available from Kyowa Interface Science Co., Ltd.).

2. Three-Dimensional Object Producing Method

The three-dimensional object of the present disclosure is produced based on biological property information indicating a biological property obtained by MRE measurement of an organism. Being produced in this way, the three-dimensional object of the present disclosure has a distribution of a strength property corresponding to the distribution of the biological property. Hence, the producing method for producing the three-dimensional object of the present disclosure based on biological property information will be described.

The object producing method of the present disclosure is not particularly limited so long as the object producing method can produce a three-dimensional object having a distribution of a strength property corresponding to the distribution of the biological property, and, for example, includes an object producing step of producing a three-dimensional object using a 3D printer based on medical 3D data of an organism. As needed, the object producing method of the present disclosure may include an obtaining step of obtaining the medical 3D data of the organism and a generating step of generating 3D data for object production based on the medical 3D data, as the steps to be performed before the object producing step. Moreover, the accuracy of a three-dimensional object produced according to the object producing method of the present disclosure can be evaluated according to a method for evaluating accuracy of a three-dimensional object of the present disclosure.

In the present disclosure, an “organism” represents at least part that constitutes a human or a living thing other than a human. The number of target organisms may be one or a plural number. The part that constitutes a human or a living thing other than a human represents, for example, a predetermined region of an organism such as a chest, or a predetermined organ in an organism.

In the present disclosure, “production of a three-dimensional object using a 3D printer based on medical 3D data of an organism” is not limited to production of a three-dimensional object by direct use of the medical 3D data, such as by inputting the medical 3D data into the 3D printer, but also includes production of a three-dimensional object by indirect use of the medical 3D data, such as by inputting 3D data for object production, which is generated based on the medical 3D data, into the 3D printer.

(1) Obtaining Step

The object producing method preferably includes an obtaining step of obtaining medical 3D data of an organism. In the present disclosure, “medical 3D data” means data generated based on at least medical image data obtained by capturing an image of an organism with a medical image capturing device and a biological property obtained by MRE measurement of the organism. That is, the obtaining step of obtaining medical 3D data of an organism includes an image capturing step of obtaining medical image data by capturing an image of an organism with a medical image capturing device, a measuring step of obtaining a biological property of an organism by MRE measurement of the organism, and a generating step of generating medical 3D data based on the medical image data and the biological property. In the present disclosure, a biological property is a measurement of a physicochemical property of an organism, such as viscoelasticity, X-ray transmittance, and conductivity.

(I) Image Capturing Step

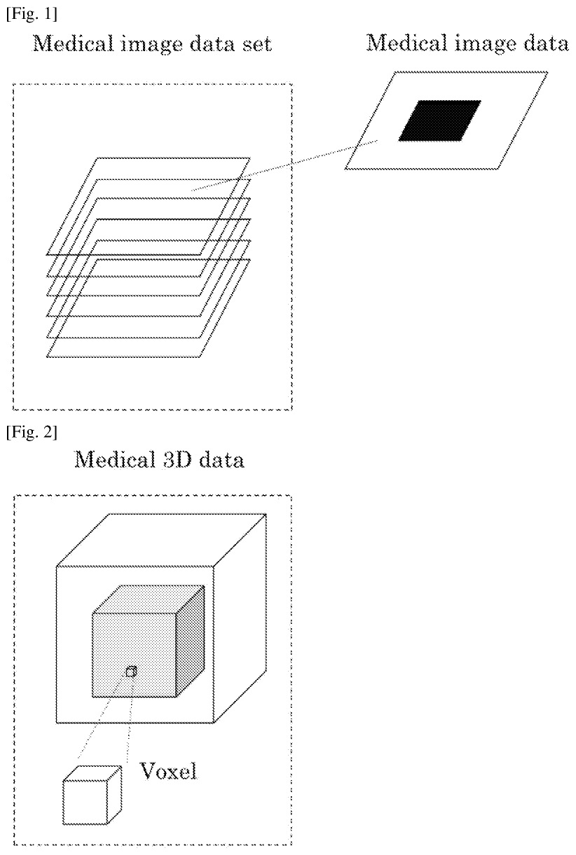

The image capturing step is a step of obtaining medical image data by capturing an image of an organism with a medical image capturing device. The medical image capturing device is also referred to as modality, and is configured to scan an organism serving as a subject and obtain medical image data. Examples of the medical image capturing device include a Computed Tomography (CT) device, a Magnetic Resonance Imaging (MRI) device, and ultrasonic diagnostic equipment. Among these devices, an MRI device is preferable because the MRI device can perform MRE measurement in the measuring step described below. The medical image capturing device performs slice tomography of capturing cross-sectional images of an organism a plurality of times. In this way, the medical image capturing device can obtain a plurality of medical image data, which are images representing cross-sections of an organism, as illustrated in FIG. 1. It is preferable that each of the plurality of medical image data (also referred to as “medical image data set”) be an image of a DICOM-format, which is an international standard relating to medical image exchange. The medical image data include image density information indicating image densities obtained by the medical image capturing device. When a CT device is used as the medical image capturing device, the image density is a CT value (X ray transmittance). When an MRI device is used as the medical image capturing device, the image density is an MRI signal value. When ultrasonic diagnostic equipment is used as the medical image capturing device, the image density is a reflection intensity.

(II) Measuring Step

The measuring step is a step of obtaining a biological (model) property by MRE measurement of an organism (model). MRE measurement is a measurement according to Magnetic Resonance Elastography (MRE). This method is a non-invasive method that measures a viscoelasticity distribution in a subject by capturing images of the subject with an MRI device while generating a shear wave inside the subject with an exciter. That is, it is preferable that the biological property obtained by MRE measurement of an organism be viscoelasticity. Examples of the method for obtaining the viscoelasticity of an organism include not only the MRE measurement method, but also an ultrasonic elastography measurement method. In this regard, information obtained by ultrasonic elastography measurement is one-dimensional information, whereas information obtained by MRE measurement is two-dimensional information. Therefore, MRE measurement is preferable because it is easier to generate 3D data. When the measuring target is a periodically moving organ such as heart and blood vessel, examples of the method for obtaining a biological property includes a method of measuring the amount of deformation of an organ due to this periodical movement and estimating the biological property from the measured value based on, for example, a dynamical model. However, the biological property obtained by this method is merely an estimated value. Therefore, this method is inferior to MRE measurement in terms of obtaining accurate information of an organism. In addition, this method can be used only when the measuring target is a periodically moving organ such as heart and blood vessel. Therefore, MRE measurement is preferable because MRE measurement can measure as targets, an organ without periodical movement and an organ of which amount of deformation due to periodical movement is difficult to measure. MRE measurement is often included in the system of an MRI device as an optional function. Hence, MRE measurement is preferable also because the image capturing step and the measuring step can be performed by the same MRI device. Incidentally, the image capturing step by an MRI device and the measuring step by an MRI device can be performed almost at the same time. It is preferable to perform these steps almost at the same time, because this makes it possible to reduce burden on the subject such as a patient, and to obtain accurate information of an organism since medical image data and a biological property are obtained almost at the same time. Particularly, the latter is an important aspect, considering that the image-capturing and measuring target is an organism having a nature that the shape and properties thereof tend to change over time. “Almost at the same time” includes, for example, a case where the period of time for the image capturing step and the period of time for the measuring step at least partially overlap, and a case where both of the image capturing step and the measuring step can be performed by use of an MRI device once. MRE measurement can be performed on an organism. Therefore, MRE measurement is preferable also because it is possible to obtain a biological property of an organ to which a blood pressure is applied in the organism.

(III) Generating Step

The generating step is a step of generating medical 3D data based on medical image data obtained in the image capturing step and the biological property obtained in the measuring step. The medical 3D data is information that includes a plurality of voxels generated based on the medical image data, voxel-by-voxel image density information indicating an image density in the medical image data and allocated to each voxel, and voxel-by-voxel biological property information indicating a biological property allocated to each voxel. In other words, the medical 3D data is information that associates voxels, image density information, and biological property information with one another.

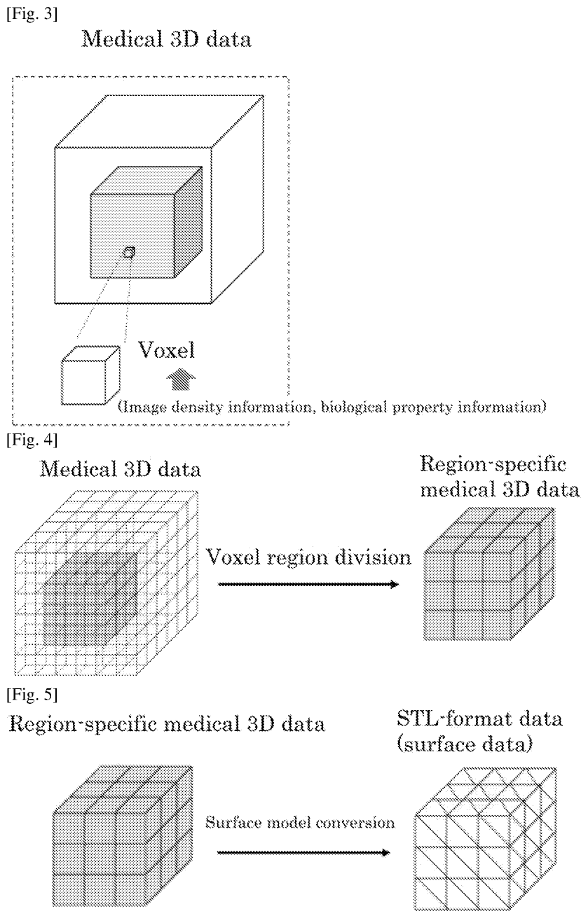

The method for generating the medical 3D data will be specifically described. First, the medical image capturing device or an image processing device relating to the medical image capturing device (both also referred to as “medical image capturing device, etc.”) generates medical 3D data for 3D image processing, from the medical image data obtained in the image capturing step. The medical 3D data is an aggregate of voxels including at least one minimum unit such as a cube as illustrated in FIG. 2, and various kinds of information can be associated with each voxel. Next, the medical image capturing device, etc. associates image density information indicating an image density in the medical image data obtained in the image capturing step with each voxel. Then, the medical image capturing device, etc. associates biological property information indicating the biological property obtained in the measuring step with each voxel. In this way, medical 3D data, which is information associating voxels, image density information, and biological property information with one another can be generated as illustrated in FIG. 3. Using the image density information, the medical 3D data can be regionally divided (segmented) in the voxel units. Voxel region division is sorting and segmentation (compartmentalization) of voxels having close image densities. This enables, for example, shape recognition, structural division, tissue analyses, and 3D image analyses of an internal structure of an organism based on the medical 3D data, and, for example, makes it possible to obtain medical 3D data of an intended organ portion (a predetermined tissue of, for example, liver) from the medical 3D data of an organism. In the present disclosure, as illustrated in FIG. 4, medical 3D data of a partial region obtained by voxel region division of the medical 3D data is referred to as region-specific medical 3D data. Region-specific medical 3D data is one concept encompassed in the medical 3D data.

(2) Generating Step

The object producing method preferably includes a generating step of generating 3D data for object production based on the medical 3D data. In the present disclosure, “3D data for object production” means data generated based on the medical 3D data and input into a 3D printer. 3D data for object production may be formed of one data or a plurality of data. Two specific examples of the method for generating 3D data for object production will be described below. However, the method for generating 3D data for object production is not limited to these examples.

(I) Generation of 3D Data for Object Production Including STL-Format Data

Generation of 3D data for object production including Standard

Triangulated Language (STL)-format data based on the medical 3D data will be described.

In this case, as illustrated in FIG. 5, region-specific medical 3D data obtained by voxel region division of the medical 3D data is converted to a surface model, to generate STL-format data corresponding to the region-specific medical 3D data. Because the STL format-data is surface data, the biological property information allocated to each voxel of the region-specific medical 3D data is lost through the surface model conversion. Hence, separately from the surface model conversion, biological property information for 3D object production including position information indicating the position of a voxel and biological property information allocated to each position information is generated based on the region-specific medical 3D data. Biological property information allocated to each position information means biological property information that has been associated with a voxel that has been present at the position indicated by the position information. That is, 3D data for object production as used herein includes STL-format data and biological property information for 3D object production.

(II) Generation of 3D Data for Object Production Including FAV-Format Data

Generation of 3D data for object production including FAbricatable Voxel (FAV)-format data based on the medical 3D data will be described.

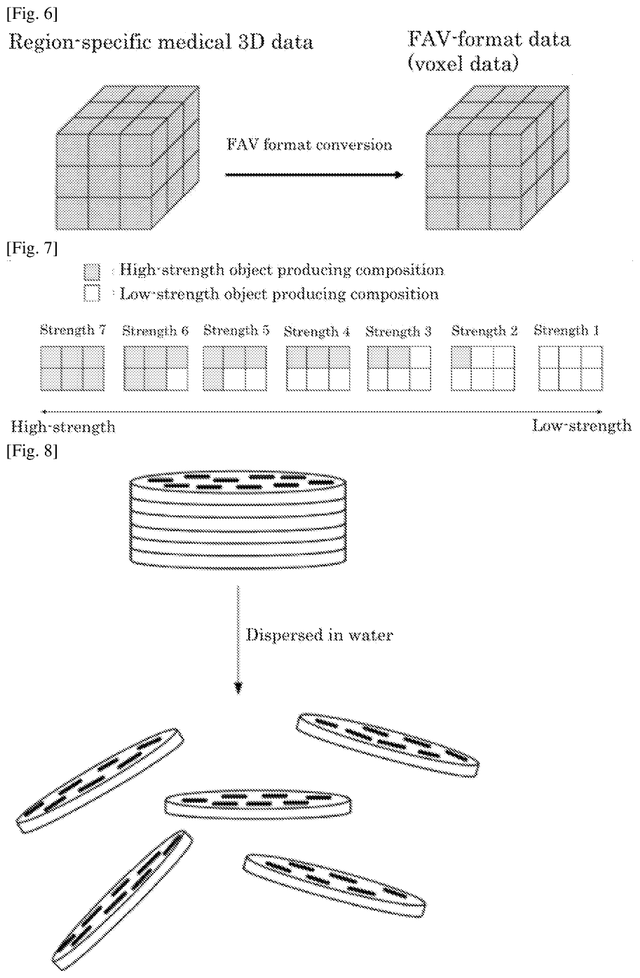

In this case, as illustrated in FIG. 6, region-specific medical 3D data obtained by voxel region division of the medical 3D data is converted to a FAV format, to generate FAV-format data corresponding to the region-specific medical 3D data. Unlike STL-format data, FAV-format data is not surface data, but is defined as voxel data. Therefore, the medical 3D data, which is voxel data to which, for example, image density information and biological property information are allocated, can be converted to FAV-format data with, for example, the image density information and the biological property information kept allocated. That is, unlike the conversion to STL-format data, advantageously, it is possible to skip the step of separately performing data processing relating to necessary information such as biological property information.

(3) Object Producing Step

The object producing method includes an object producing step of producing a three-dimensional object using a 3D printer based on the medical 3D data of an organism. As described above, in the present disclosure, “production of a three-dimensional object using a 3D printer based on the medical 3D data of an organism” is not limited to production of a three-dimensional object by direct use of the medical 3D data, such as by inputting the medical 3D data into the 3D printer, but also includes production of a three-dimensional object by indirect use of the medical 3D data, such as by inputting 3D data for object production, which is generated based on the medical 3D data, into the 3D printer. The object producing step will be described below, regarding production of a three-dimensional object by inputting 3D data for object production generated based on the medical 3D data into a 3D printer, as an example.

First, a case where the above-described 3D data for object production including STL-format data is input into the 3D printer will be described. In this case, the 3D data for object production includes STL-format data and biological property information for 3D object production. These data may be input simultaneously or separately.

The 3D printer converts the input STL-format data to a 2D image data set for printing. The 2D image data set for printing is a data set including a plurality of 2D image data for printing. The 2D image data for printing are two-dimensional slice data obtained by slicing the STL-format data at intervals defined by the resolution of the 3D printer in the Z axis direction. Next, the 3D printer allocates strength property information indicating a strength property to each region of the 2D image data for printing based on the input biological property information for 3D object production. The 3D printer repeats a step of discharging a plurality of kinds of object producing compositions to predetermined regions in predetermined discharging amounts based on the 2D image data for printing and the strength property information for each region in the 2D image data for printing, and curing the object producing compositions. As a result, a three-dimensional object having a distribution of the strength property corresponding to the distribution of the biological property can be produced. Here, in the present disclosure, the “strength property” means a property of a three-dimensional object produced with a 3D printer, and specifically means viscoelasticity because it is a property corresponding to the biological property.

Next, a case where the above-described 3D data for object production including FAV-format data is input into the 3D printer will be described.

The 3D printer converts the input FAV-format data to a 2D image data set for printing, which is the same slice data as described above except that strength property information indicating a strength property of each region is allocated to each 2D image data for printing included in the 2D image data set for printing, based on the biological property information included in the FAV-format data. The 3D printer repeats a step of discharging a plurality of kinds of object producing compositions to predetermined regions in predetermined discharging amounts based on the 2D image data for printing and the strength property information for each region in the 2D image data for printing, and curing the object producing compositions. As a result, a three-dimensional object having a distribution of the strength property corresponding to the distribution of the biological property can be produced.

Examples of the 3D printer used in the object producing step include a printer that can control the strength property of a three-dimensional object to be produced, region by region of the three-dimensional object, by discharging a plurality of kinds of object producing compositions to predetermined regions in predetermined discharging amounts. Specific examples of such a 3D printer include a 3D printer of a material jetting type (MJ type) configured to discharge the object producing compositions from inkjet heads.

Examples of the method for bringing correspondence between the strength property and the biological property as described above using a 3D printer include a method of using a 3D printer that can express the strength property by gradation. Hence, an example of this method will be described.

First, a high-strength object producing composition that can produce a three-dimensional object having a high strength property and a low-strength object producing composition that can produce a three-dimensional object having a low strength property are prepared. For each of these object producing compositions, at least one kind of a composition or a plurality of kinds of compositions may be used. It is preferable that the strength property of a three-dimensional object to be produced with the high-strength object producing composition be greater than or equal to the maximum biological property value in the organism. It is preferable that the strength property of a three-dimensional object to be produced with the low-strength object producing composition be less than or equal to the minimum biological property value in the organism.

Next, the strength property of the three-dimensional object is controlled based on the pattern according to which the object producing compositions are arranged in order for a certain grid region to be filled with the object producing compositions. For example, as illustrated in FIG. 7, when expression of the strength property by seven gradations is needed, seven-gradation strength can be expressed by combination patterns according to which a grid, whose minimum unit is formed of six segments, is filled with the high-strength object producing composition and the low-strength object producing composition. By increasing the number of segments in a unit region of the grid, it is possible to realize various gradation expressions. A strength distribution in a horizontal direction can be set by arrangement of the compositions in the XY plane, and a strength distribution in the vertical direction can be set by arrangement of the compositions in the Z direction. This makes it possible to produce a three-dimensional object having a distribution of the strength property corresponding to the distribution of the biological property. That is, in the present disclosure, “a distribution of the strength property corresponding to the distribution of the biological property” is not limited to a distribution in which a strength property is equal to a biological property at the corresponding position, but also includes a distribution in which a strength property realized by gradation expression is approximate to a biological property at the corresponding position. “Approximate” means, for example, a difference of within 5% between the strength property (the same as a model property described below) and the biological property. In the present disclosure, “gradations” mean respective arrangement patterns, each of which is formed by the plurality of kinds of object producing compositions being arranged, and which define different strength properties in a cured product.

This method is efficient with voxel formats such as FAV, because voxel formats enable voxel expression. The number of liquid droplets of the object producing compositions for filling the unit segment is optional.

By previously knowing the strength properties of the gradations that can be expressed by a 3D printer by, for example, MRE measurement, it is possible to more accurately produce a three-dimensional object having a distribution of the strength property corresponding to the distribution of the biological property.

3. Method for Evaluating Accuracy of Three-Dimensional Object

The method for evaluating accuracy of a three-dimensional object of the present disclosure includes an evaluating step of evaluating accuracy of a three-dimensional object imitating an organism with respect to the organism based on biological property information indicating a biological property and three-dimensional object property information indicating a property of the three-dimensional object. The evaluating step is preferably a step of evaluating accuracy of a three-dimensional object imitating an organism with respect to the organism based on biological property information included in the medical 3D data and three-dimensional object property information included in three-dimensional object 3D data. This is because, as described above, it is preferable to obtain the biological property information as information to be included in the medical 3D data, and obtain three-dimensional object property information as information to be included in three-dimensional object 3D data.

Accuracy of a three-dimensional object indicates how correctly a three-dimensional reproduces an organism. Accuracy of a three-dimensional object can be calculated based on accuracy of a property and accuracy of the shape.

The accuracy of a property indicates how correctly a property of a three-dimensional object reproduces a property of an organism.

The accuracy of the shape indicates how correctly the shape of a three-dimensional object reproduces the shape of an organism.

The evaluating step includes a property evaluating step and a shape evaluating step.

The property evaluating step is a step of evaluating how correctly a property of a three-dimensional object reproduces a property of organism, and can evaluate the accuracy by calculating a concrete difference between biological property information and three-dimensional object property information using a computer and comparing a threshold with the calculated difference to judge which is the greater than the other. The property evaluating step may also judge presence or absence of any difference between the biological property information and the three-dimensional object property information.

The evaluating step may evaluate the accuracy based on the full data of the biological property information and the three-dimensional object property information, or may evaluate the accuracy based on partial data of the biological property information and the three-dimensional object property information or on a voxel-by-voxel basis. This step may be performed by a human or by an information processing device such as a personal computer.

In the evaluating step, images of predetermined cross-sections may be generated based on the biological property information and the three-dimensional object property information, and the images may be displayed by superimposition of the images or side-by-side arrangement of the images, or the images may be printed so that a judge can superimpose the images or arrange the images side by side and observe the images. The method for evaluating accuracy of a three-dimensional object of the present disclosure includes at least a property evaluating step of evaluating accuracy of a three-dimensional object based on property information and, as needed, may further include a shape evaluating step of evaluating accuracy of a three-dimensional object based on shape information.

First, a method for evaluating accuracy of a produced three-dimensional object based on biological property information and three-dimensional object property information will be described.

Examples of the method for evaluating accuracy of a three-dimensional object include a method of evaluating accuracy by judging whether the distribution of the biological property and the distribution of a property of a three-dimensional object represent almost the same distribution, and a method of evaluating accuracy by judging whether an organism and a three-dimensional object have almost the same biological property and three-dimensional object property at corresponding parts.

Examples of the corresponding parts of an organism and a three-dimensional object include: a part of the organism and a part of the three-dimensional object located at almost the same coordinates when coordinate systems are allocated to the organism and the three-dimensional object on the same basis; and a part having a specific structure in an organism (e.g., a tumor) and a part in a three-dimensional object imitating the part having the specific structure in the organism (e.g., a part imitating the tumor). The latter is preferable. In the latter case, for example, ten positions are selected from each of the part having the specific structure in the organism and the part in the three-dimensional object imitating the part having the specific structure in the organism, to calculate the average of the biological property and the average of the three-dimensional object property in the these parts, and evaluate accuracy of the three-dimensional object based on these averages.

Examples of the criterion for judging whether the organism and the three-dimensional object have almost the same biological property and three-dimensional object property at corresponding parts include a difference of within 5% between the three-dimensional object property and the biological property at the corresponding parts. The difference is not limited to within 5% but may be appropriately selected depending on the degree of accuracy needed, and may be, for example, within 50%, within 40%, within 30%, within 20%, and within 10% but is preferably as small as possible because it is possible to evaluate that the three-dimensional object is highly accurate. It is possible to realize a three-dimensional object having a difference of within 5%, by performing the image capturing step by an MRI device and the measuring step by an MRI device almost at the same time. This is because, as described above, medical image data and a biological property are obtained almost at the same time and accurate information of the organism can be obtained as a result. “Almost at the same time” includes, for example, a case where the period of time for the image capturing step and the period of time for the measuring step at least partially overlap, and a case where both of the image capturing step and the measuring step can be performed by use of an MRI device once.

In order to compare the biological property information and the three-dimensional object property information of the corresponding parts of the organism and the three-dimensional object as described above, it is preferable to have information indicating the position at which the biological property is measured in the organism and information indicating the position at which the three-dimensional object property is measured in the three-dimensional object. Hence, when evaluating accuracy of the three-dimensional object, it is preferable to use medical 3D data, which is information associating voxels, image density information, and biological property information and three-dimensional object 3D data, which is information associating voxels, image density information, and three-dimensional object property information and compare the biological property information and the three-dimensional object property information included in these data. This is because voxels correspond to the information indicating the positions. The three-dimensional object 3D data is data obtained in the same manner as obtaining the medical 3D data, and represents data generated based on at least model image data obtained by capturing an image of the three-dimensional object with a medical image capturing device and a three-dimensional object property obtained by MRE measurement of the three-dimensional object.

In the evaluating step, a three-dimensional object property obtained by MRE measurement of the three-dimensional object is used. This suggests that it is preferable that the material constituting the three-dimensional object contain as a main component, water suitable for MRE measurement in which a hydrogen nucleus is used as the signal source. That is, it is preferable that the material constituting the three-dimensional object contain the hydrogel described above.

4. Method for Producing Three-Dimensional Object Using 3D Printer

As an example of the method for producing a three-dimensional object with a 3D printer (hereinafter, may also be referred to as “object producing apparatus”), a method for producing a three-dimensional object using the three-dimensional hydrogel object producing composition described above in a 3D printer of a material jetting type will be described.

The method for producing a three-dimensional object by a material jetting method includes a liquid film forming step of applying a liquid droplet of the three-dimensional hydrogel object producing composition to form a liquid film, and a curing step of curing the liquid film of the three-dimensional hydrogel object producing composition, and repeats the liquid film forming step and the curing step sequentially. The three-dimensional object producing method may include, as needed, a support producing step of producing a support for supporting a three-dimensional object and other steps.

A three-dimensional object producing apparatus of a material jetting type includes a storing unit storing the three-dimensional hydrogel object producing composition, a liquid film forming unit configured to apply a liquid droplet of the three-dimensional hydrogel object producing composition stored, to form a liquid film, and a curing unit configured to cure the liquid film of the three-dimensional hydrogel object producing composition, and repeats the formation of a liquid film by the liquid film forming unit and curing by the curing unit sequentially.

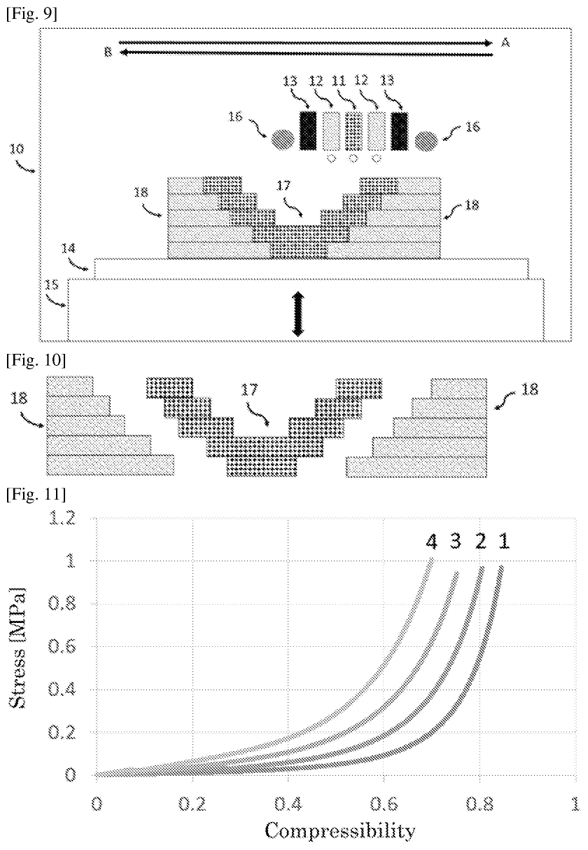

The material jetting method will be described with reference to FIG. 9 and FIG. 10. FIG. 9 is an exemplary view illustrating an example of the three-dimensional object producing apparatus. FIG. 10 is an exemplary view illustrating an example of a three-dimensional object detached from supports. The three-dimensional object producing apparatus 10 of a material jetting type illustrated in FIG. 9 uses head units in which inkjet heads are arranged, and is configured to cause a three-dimensional hydrogel object producing composition discharging head unit 11 to discharge a three-dimensional hydrogel object producing composition stored in a three-dimensional hydrogel object producing composition storing container and cause support producing composition discharging head units 12 to discharge a support producing composition stored in a support producing composition storing container to an object support substrate 14, and cause adjacent ultraviolet irradiators 13 to cure the three-dimensional hydrogel object producing composition and the support producing composition, to laminate layers. The support producing composition is a liquid composition that cures in response to irradiation with active energy rays such as light or heat, to produce a support for supporting a three-dimensional object. Examples of the support producing composition include acrylic-based materials. The object producing apparatus 10 may include a smoothing member 16 configured to smooth the three-dimensional hydrogel object producing composition discharged.

The object producing apparatus 10 is configured to laminate layers while lowering a stage 15 in accordance with the number of layers laminated, in order to keep the three-dimensional hydrogel object producing composition discharging head unit 11, the support producing composition discharging head units 12, and the ultraviolet irradiators 13 at a constant gap from the three-dimensional object (hydrogel) 17 and the supports (support materials) 18.

The ultraviolet irradiators 13 of the object producing apparatus 10 are used in the travels in the directions of both of the arrows A and B. Heat generated along with ultraviolet irradiation smooths the surfaces of the laminated layers, and the dimensional stability of the three-dimensional object 17 is improved as a result.

After object production by the object producing apparatus 10 is completed, the three-dimensional object 17 and the supports 18 are drawn in the horizontal direction as illustrated in FIG. 10. As a result, the supports 18 are detached as integrated bodies, and the three-dimensional object 17 can be taken out easily.

(1) Liquid Film Forming Step

The method for applying the three-dimensional hydrogel object producing composition in the liquid film forming step is not particularly limited and may be appropriately selected depending on the intended purpose so long as the method can apply a liquid droplet to an intended position with a suitable accuracy. A known liquid droplet discharging method can be used. Specific examples of the liquid droplet discharging method include a dispenser method, a spray method, and an inkjet method. The inkjet method is preferable.

The volume of a liquid droplet of the three-dimensional hydrogel object producing composition is preferably 2 pL or greater but 60 pL or less and more preferably 15 pL or greater but 30 pL or less. When the volume of a liquid droplet is 2 pL or greater, discharging stability can be improved. When the volume of a liquid droplet is 60 pL or less, for example, discharging nozzles for object production can be easily filled with the three-dimensional hydrogel object producing composition.

(2) Curing Step

Examples of the curing unit configured to cure the liquid film of the three-dimensional hydrogel object producing composition in the curing step include ultraviolet (UV) irradiation lamps, and electron beams. Examples of the kinds of the ultraviolet (UV) irradiation lamps include high-pressure mercury lamps, ultrahigh-pressure mercury lamps, and metal halides.

As the curing unit for curing the three-dimensional hydrogel object producing composition, an Ultra Violet-Light Emitting Diode (UV-LED) can be suitably used. The emission wavelength of the LED is not particularly limited and is typically, for example, 365 nm, 375 nm, 385 nm, 395 nm, and 405 nm. Considering influences of colors on the object, shorter wavelength emission is more advantageous for increasing absorption by an initiator. Moreover, the UV-LED generates a lower thermal energy during curing and can save thermal damage on the hydrogel, compared with ultraviolet irradiation lamps commonly used (e.g., high-pressure mercury lamps, ultrahigh-pressure mercury lamps, and metal halides) and electron beams. This effect is particularly remarkable for hydrogels to be produced with the three-dimensional hydrogel object producing composition, because such hydrogels are used in a state of containing water.

The three-dimensional object producing method includes a liquid film forming step of applying a liquid droplet of the three-dimensional hydrogel object producing composition to form a liquid film and a curing step of curing the liquid film of the three-dimensional hydrogel object producing composition, and repeats the liquid film forming step and the curing step sequentially. The number of times of repetition is not particularly limited and may be appropriately selected depending on, for example, the size and shape of the three-dimensional object to be produced. The average thickness per cured layer is preferably 10 micrometers or greater but 50 micrometers or less. When the average thickness is 10 micrometers or greater but 50 micrometers or less, it is possible to produce an object accurately while suppressing peel or detachment.

(3) Support Producing Step

The support producing composition used in the support producing step is a liquid composition that cures in response to irradiation with active energy rays such as light or heat, to produce a support for supporting a three-dimensional object. The support producing composition is compositionally different from the three-dimensional hydrogel object producing composition. Specifically, it is preferable that the support producing composition contain, for example, a curable material and a polymerization initiator, and be free of water and a mineral. The curable material is a compound that undergoes a polymerization reaction and cures in response to, for example, irradiation with active energy rays (e.g., ultraviolet rays and electron beams) and heating, and examples of the curable material include active-energy-ray-curable compounds and thermosetting compounds. Among these curable materials, materials that are liquid at normal temperature are preferable.

The support producing composition is applied to a position different from the position to which the three-dimensional hydrogel object producing composition is applied. This means that the support producing composition and the three-dimensional hydrogel object producing composition do not overlap with each other. The support producing composition and the three-dimensional hydrogel object producing composition may adjoin each other.

Examples of the method for applying the support producing composition include the same as the methods for applying the three-dimensional hydrogel object producing composition.

(4) Other Steps

Examples of the other steps include a step of smoothing a liquid film, a detaching step, a polishing step of polishing a three-dimensional object, and a washing step of washing a three-dimensional object. It is preferable to include the step of smoothing a liquid film. This is because the liquid film formed in the liquid film forming step does not always have the intended film thickness (layer thickness) at all positions. For example, when forming a liquid film by an inkjet method, there may occur, for example, discharging failure or inter-dot height difference, making it difficult to form a highly accurate three-dimensional object. To such a problem, conceivable methods include a method of mechanically smoothing (leveling) a liquid film after formed, a method of mechanically scraping away a hydrogel thin film obtained through curing of the liquid film, and a method of sensing the degree of smoothness and adjusting the amount of film formation on a dot level during lamination of the next layer. When producing a three-dimensional hydrogel object as an organ model, the method of mechanically leveling a liquid film is preferable as the smoothing method because the hydrogel serving as the material constituting the organ model is relatively soft. Examples of the mechanical smoothing method include a leveling method using a blade-shaped member and a leveling method using a roller-shaped member.

(5) Method for producing three-dimensional object including parts having different strength properties.

Next, as a more specific description of the three-dimensional object producing method, an example of the method for producing a three-dimensional object including parts having different strength properties will be described. The following description will be made taking as an example, an embodiment where two kinds of compositionally different three-dimensional hydrogel object producing compositions are used. However, this embodiment is non-limiting. A person ordinarily skilled in the art would easily understand another embodiment (for example, an embodiment where three or more kinds of three-dimensional hydrogel object producing compositions are used) based on the following description.

The method for producing a three-dimensional object including parts having different strength properties includes a liquid film forming step of separately applying liquid droplets of a plurality of compositionally different three-dimensional hydrogel object producing compositions to form a liquid film including a plurality of compositionally different regions, and a curing step of curing the liquid film, and repeats the liquid film forming step and the curing step sequentially.

The object producing method described above employs a three-dimensional object producing apparatus that includes storing units separately storing a plurality of compositionally different three-dimensional hydrogel object producing compositions, a liquid film forming unit configured to separately apply liquid droplets of the plurality of compositionally different three-dimensional hydrogel object producing compositions stored, to form a liquid film including a plurality of compositionally different regions, and a curing unit configured to cure the liquid film, where the three-dimensional object producing apparatus repeats the formation of a liquid film by the liquid film forming unit and the curing by the curing unit sequentially.

Specifically, using a first three-dimensional hydrogel object producing composition and a second three-dimensional hydrogel object producing composition compositionally different from the first three-dimensional hydrogel object producing composition, a liquid film continuously including a plurality of compositionally different regions is formed based on control on the positions to which and the amounts by which liquid droplets of the respective three-dimensional hydrogel object producing compositions are applied. The first three-dimensional hydrogel object producing composition is an example of the high-strength object producing composition described above, and the second three-dimensional hydrogel object producing composition is an example of the low-strength object producing composition. Next, the liquid film is cured, to form a cured film for one layer continuously including the regions described above. Subsequently, the formation of a liquid film and the curing are repeated sequentially, to laminate cured films and produce a three-dimensional object continuously including a plurality of parts having different strength properties. The plurality of parts having different strength properties in the three-dimensional object may be present by having different strength properties in a cured film for one layer or may be present by having different strength properties between cured films.

5. Use of Three-Dimensional Object

(1) General-Purpose Use or Individual-Specific Use

Examples of the use of the three-dimensional object include a tissue model imitating a tissue of an organism. A human tissue model is preferable. A tissue means a functional organ constituting an organism. 2Therefore, the tissue is not limited to internal organs, but also encompass all kinds of organs that constitute an organism, such as bones, skin, and blood vessels. An organ model is preferable as the tissue model.

The use of the three-dimensional object is roughly classified into two types. One is a three-dimensional object in an individual-specific use, and the other is a three-dimensional object in a general-purpose use.

The three-dimensional object in an individual-specific use is a three-dimensional object used for, for example, informed consent, preoperative simulations, and operative method training in the medical settings. In this case, the three-dimensional object has the shape and properties of the organ that includes the affected part of the target individual patient. Also in this case, it is preferable that the three-dimensional object be a model that is produced based on data of the individual patient and reproduces the affected part.

The method for producing a three-dimensional object in an individual-specific use includes an object producing step of producing a three-dimensional object in an individual-specific use using a 3D printer based on individual medical 3D data, which is medical 3D data of one organism. The object producing method including such an object producing step may include, as the steps to be performed before the object producing step, an obtaining step of obtaining individual medical 3D data, and a generating step of generating 3D data for individual object production, which is 3D data for object production, based on the individual medical 3D data.

The obtaining step further includes an image capturing step of capturing an image of one organism with a medical image capturing device to obtain individual medical image data, which is medical image data, a measuring step of obtaining an individual biological property, which is a biological property, by MRE measurement of one organism, and a generating step of generating individual medical 3D data based on the individual medical image data and the individual biological property.

The three-dimensional object in a general-purpose use is a three-dimensional object used for, for example, performance validation, calibration, and training in medical device development, and structure confirmation in the educational settings. In this case, the three-dimensional object has an average shape and average properties of a target organ. Also in this case, it is preferable that the three-dimensional object be a model that is produced based mainly on data of a healthy person and based on data of a single person or average data of a plurality of persons.

The method for producing a three-dimensional object in a general-purpose use includes an object producing step of producing a three-dimensional object in a general-purpose use using a 3D printer based on average medical 3D data obtained by averaging individual medical 3D data, which are medical 3D data of a plurality of organisms. When the average medical 3D data includes information obtained by MRE measurement, it is meant that the average medical 3D data includes the biological property information of the present disclosure. The object producing method including such an object producing step may include, as the steps to be performed before the object producing step, an obtaining step of obtaining a plurality of individual medical 3D data and averaging the obtained plurality of individual medical 3D data to obtain average medical 3D data, and a generating step of generating 3D data for average object production, which is 3D data for object production, based on the average medical 3D data.

The obtaining step further includes an image capturing step of capturing images of a plurality of organisms with a medical image capturing device to obtain a plurality of individual medical image data, which are medical image data, a measuring step of obtaining a plurality of individual biological properties, which are a biological property, by MRE measurement of a plurality of organisms, a generating step of generating a plurality of individual medical 3D data based on the individual medical image data and the individual biological properties of the respective organisms, and an averaging step of averaging the plurality of individual medical 3D data to obtain average medical 3D data. The average medical 3D data includes average voxels, average image density information, and average biological property information obtained by averaging individual voxels, individual image density information, and individual biological property information included in the plurality of individual medical 3D data. As the averaging method, an appropriate known method can be used.

(2) Training Use

It is preferable to use a three-dimensional object for operative trainings. Because the three-dimensional object of the present disclosure reproduces the shape and properties of an organism, the three-dimensional object alone is effective for, for example, operative techniques training. However, for trainings (i.e., trainings intended for being acquainted with the procedure, and approach to the affected part) and simulations of the entire operation, it is preferable to use a training system that includes the three-dimensional object of the present disclosure and a housing that houses the three-dimensional object. The housing is not particularly limited. For example, the configuration and shape of the housing may be appropriately selected depending on the training use. For example, for endoscope training, it is preferable that the three-dimensional object be unseeable from outside. Therefore, the housing is produced with a material that eliminates or reduces visibility of an organ to be housed, and the three-dimensional object is disposed inside the housing. Here, a shape of the housing having a structure imitating an organism (e.g., the structure and skeleton of a human body) is effective and preferable. A wide variety of such housings are commercially available, from housings having simple shapes such as training boxes for laparoscope to housings having complicated shapes such as ones including ribs inside. These commercially available products may be appropriately used.

(3) Use as Human Organ Model

When an object is used as a human organ model, the content of water among materials constituting the object, such as a hydrogel is preferably 70% by mass or greater but 85% by mass or less relative to the total amount of the organ model. When the content of water is 70% by mass or greater but 85% by mass or less, the water content in the human organ model can be equal or similar to the water content in the target actual human organ, and the human organ model can become suitable for use. Specifically, the content of water in a human heart model is preferably about 80% by mass, the content of water in a human kidney model is preferably about 83% by mass, and the content of water in a human brain or bowel model is preferably about 75% by mass. Accordingly, the content of water in an organ model is more preferably 75% by mass or greater but 83% by mass or less relative to the total amount of the organ model.

### EXAMPLES

The present disclosure will be described below by way of Examples. The present disclosure should not be construed as being limited to these Examples.

<Production of a High-Strength Object Producing Composition A>

First, ion-exchanged water was subjected to pressure reducing deaeration for 30 minutes to prepare pure water. To the pure water (580.0 parts by mass) under stirring, synthetic hectorite (LAPONITE RD, obtained from Byk Additives & Instruments, Inc.) serving as a water-swellable clay mineral (67.0 parts by mass) was added little by little, and the resultant was further stirred to produce a mixture liquid. Next, to the mixture liquid, etidronic acid (obtained from Tokyo Chemical Industry Co., Ltd.) (5.0 parts by mass) was added as a dispersant of the synthetic hectorite, to obtain a dispersion liquid.

Next, to the obtained dispersion liquid, dimethyl acrylamide (DMAA, obtained from Tokyo Chemical Industry Co., Ltd.) (262.0 parts by mass) having passed through an active alumina column to remove any polymerization initiator was added as a monomer. To the resultant, N,N′-methylene bisacrylamide (MBAA, obtained from Tokyo Chemical Industry Co., Ltd.) (2.4 parts by mass) and polyethylene glycol diacrylate (A-400, obtained from Shin-Nakamura Chemical Co., Ltd.) (8.0 parts by mass) were added as cross-linking agents. To the resultant, glycerin (obtained from Sakamoto Yakuhin Kogyo Co., Ltd.) (300.0 parts by mass) was added as an anti-drying agent, and the resultant was mixed.

Next, to the resultant, N,N,N′,N′-tetramethyl ethylene diamine (TEMED, obtained from Tokyo Chemical Industry Co., Ltd.) (6.7 parts by mass) was added as a polymerization accelerator. To the resultant, EMULGEN LS-106 (obtained from Kao Corporation) (5.3 parts by mass) was added as a surfactant, and the resultant was mixed.

Next, to the resultant under cooling in an ice bath, a solution (12.3 parts by mass) of a photopolymerization initiator (IRGACURE 184, obtained from BASF GmbH) in 4% by mass of methanol was added. The resultant was stirred and mixed and subsequently subjected to pressure reducing deaeration for 20 minutes. Successively, the resultant was filtrated to remove, for example, impurities, to obtain a homogeneous high-strength object producing composition A.

—Measurement of Viscoelasticity of Cured Product of High-Strength Object Producing Composition A—

First, a hydrogel was produced using the high-strength object producing composition A. Specifically, a container having a size of 31 mm×31 mm (with a thickness of 10 mm) was prepared and filled with the high-strength object producing composition A. Then, the high-strength object producing composition A was cured using an ultraviolet irradiator (obtained from Ushio Inc., SPOT CURE SP5-250DB). The irradiation conditions include a wavelength of 365 nm, an irradiation intensity of 350 mJ/cm², and an irradiation time of 60 seconds.

Next, the physical properties of the produced hydrogel were measured with a rheometer. As a result, the storage modulus was 8,320 Pa and the loss modulus was 2,540 Pa.

<Production of Low-Strength Object Producing Composition B>

First, ion-exchanged water was subjected to pressure reducing deaeration for 30 minutes to prepare pure water. To the pure water (580.0 parts by mass) under stirring, synthetic hectorite (LAPONITE RD, obtained from Byk Additives & Instruments, Inc.) serving as a water-swellable clay mineral (67.0 parts by mass) was added little by little, and the resultant was further stirred to produce a mixture liquid. Next, to the mixture liquid, etidronic acid (obtained from Tokyo Chemical Industry Co., Ltd.) (5.0 parts by mass) was added as a dispersant of the synthetic hectorite, to obtain a dispersion liquid.

Next, to the obtained dispersion liquid, dimethyl acrylamide (DMAA, obtained from Tokyo Chemical Industry Co., Ltd.) (262.0 parts by mass) having passed through an active alumina column to remove any polymerization initiator was added as a monomer. To the resultant, N,N′-methylene bisacrylamide (MBAA, obtained from Tokyo Chemical Industry Co., Ltd.) (0.6 parts by mass) and polyethylene glycol diacrylate (A-400, obtained from Shin-Nakamura Chemical Co., Ltd.) (2.0 parts by mass) were added as cross-linking agents. To the resultant, glycerin (obtained from Sakamoto Yakuhin Kogyo Co., Ltd.) (300.0 parts by mass) was added as an anti-drying agent, and the resultant was mixed.

Next, to the resultant, N,N,N′,N′-tetramethyl ethylene diamine (TEMED, obtained from Tokyo Chemical Industry Co., Ltd.) (6.7 parts by mass) was added as a polymerization accelerator. To the resultant, EMULGEN LS-106 (obtained from Kao Corporation) (5.3 parts by mass) was added as a surfactant, and the resultant was mixed. Next, to the resultant under cooling in an ice bath, a solution (12.3 parts by mass) of a photopolymerization initiator (IRGACURE 184, obtained from BASF GmbH) in 4% by mass of methanol was added. The resultant was stirred and mixed and subsequently subjected to pressure reducing deaeration for 20 minutes. Successively, the resultant was filtrated to remove, for example, impurities, to obtain a homogeneous low-strength object producing composition B.

—Measurement of Viscoelasticity of Cured Product of Low-Strength Object Producing Composition B—

First, a hydrogel was produced using the low-strength object producing composition B. Specifically, a container having a size of 31 mm×31 mm (with a thickness of 10 mm) was prepared and filled with the low-strength object producing composition B. Then, the low-strength object producing composition B was cured using an ultraviolet irradiator (obtained from Ushio Inc., SPOT CURE SP5-250DB). The irradiation conditions include a wavelength of 365 nm, an irradiation intensity of 350 mJ/cm², and an irradiation time of 60 seconds.

Next, the physical properties of the produced hydrogel were measured with a rheometer. As a result, the storage modulus was 4,415 Pa and the loss modulus was 3,104 Pa.

<Evaluation of Young's Modulus>

Hydrogel samples 1 to 4 were produced using the high-strength object producing composition A and the low-strength object producing composition B. Specifically, a container having a size of 31 mm×31 mm (with a thickness of 10 mm) was prepared and filled with a mixture of the high-strength object producing composition A and the low-strength object producing composition B at the mass ratio presented in Table 1 below. The mixture was cured using an ultraviolet irradiator (obtained from Ushio Inc., SPOT CURE SP5-250DB), to produce samples 1 to 4. The irradiation conditions include a wavelength of 365 nm and a cumulative optical exposure of 350 mJ/cm². Next, the compressive strength of each sample was measured. The compressive strength was measured using an autograph AG-1 (obtained from Shimadzu Corporation) and a load cell of 1 MPa. The result is plotted in FIG. 11. The Young's modulus was calculated based on the slope of the plot in FIG. 11, and is presented in Table 1 below.

As plotted or presented in FIG. 11 and Table 1, it was possible to control a physical property (Young's modulus) of the hydrogels based on the mass ratio between the high-strength object producing composition A and the low-strength object producing composition B.

TABLE 1

| | Mass ratio in mixture | | Young's |
|---|---|---|---|
| | High strength object | Low strength object | (MPa) |
| Sample | producing composition A | producing composition B | modulus |
| 1 | 0 | 100 | 0.06 |
| 2 | 38 | 67 | 0.15 |
| 3 | 67 | 33 | 0.19 |
| 4 | 100 | 0 | 0.38 |

<Evaluation of Viscoelasticity>

First, hydrogel samples 5 to 8 were produced using the high-strength object producing composition A and the low-strength object producing composition B. Specifically, a container made in the shape of a dumbbell No. 3 (JIS K 7139) was prepared and filled with a mixture of the high-strength object producing composition A and the low-strength object producing composition B at the mass ratio presented in Table 2 below. The mixture was cured using an ultraviolet irradiator (obtained from Ushio Inc., SPOT CURE SP5-250DB), to produce samples 5 to 8. The irradiation conditions include a wavelength of 365 nm and a cumulative optical exposure of 350 mJ/cm².

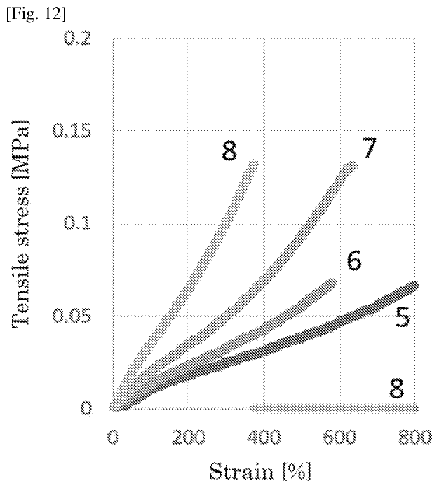

Next, each sample was subjected to a tensile test. The tensile test was performed using an autograph AG-1 (obtained from Shimadzu Corporation), and stress was measured with a force gauge. The result is plotted in FIG. 12.

As plotted in FIG. 12, it was possible to control a physical property (viscoelasticity) of the hydrogels based on the mass ratio between the high-strength object producing composition A and the low-strength object producing composition B.

TABLE 2

Mass ratio in mixture

| | High strength object | Low-strength object |
|---|---|---|
| Sample | producing composition A | producing composition B |
| 5 | 0 | 100 |
| 6 | 38 | 67 |
| 7 | 67 | 38 |
| 8 | 100 | 0 |

<Obtainment of Medical 3D Data 1>

Using an MRI device capable of performing MRE measurement, medical 3D data 1 including a liver part of a patient suffering from fatty liver was obtained. The medical 3D data 1 was generated based on medical image data obtained by capturing an image of the patient with the MRI device and a biological property obtained by MRE measurement of the patient. Specifically, the medical 3D data 1 includes a plurality of voxels generated based on the medical image data, image density information indicating an image density (MRI signal value) in the medical image data and allocated to each voxel, and biological property information indicating a biological property (viscoelasticity) allocated to each voxel.

The biological property (viscoelasticity) of the liver part obtained by MRE measurement was within the ranges of the viscoelasticity of a cured product of the high-strength object producing composition A and the viscoelasticity of a cured product of the low-strength object producing composition B at any local parts of the liver part.

<Generation of 3D Data 1 for Object Production>

The medical 3D data 1 was subjected to voxel region division, to obtain region-specific medical 3D data 1 representing the liver part. Next, the region-specific medical 3D data 1 was converted to a FAV format, to generate FAV-format 3D data 1 for object production.

<Object Production Example of Liver Model 1>

The high-strength object producing composition A and the low-strength object producing composition B were loaded into a 3D printer of a material jetting type as illustrated in FIG. 9 capable of expressing strength property gradations, and filled into inkjet heads of the 3D printer. Next, the 3D data 1 for object production was input into the 3D printer, and a liver model 1 having a distribution of a strength property corresponding to the distribution of the biological property was produced based on the 3D data 1 for object production. Specifically, the respective object producing compositions were discharged from the inkjet heads, and formation of a liquid film and curing were repeated sequentially, to produce the model.

<Production of Object Producing Composition C>

An object producing composition C was obtained in the same manner as in Production of high-strength object producing composition A, except that a solution of an initiator (potassium persulfate) in 4% by mass of water was used instead of the solution of a photopolymerization initiator (IRGACURE 184, obtained from BASF GmbH) in 4% by mass of methanol.

<Object Production Example of Liver Model 2>

A casting mold imitating the liver part of a patient was produced with a 3D printer (FORM 2, obtained from Formlabs Inc.) based on the 3D data for object production. Next, the object producing composition C was injected into the casting mold and cured by being maintained at room temperature for 24 hours, to obtain a liver model 2.

<Obtainment of Three-Dimensional Object 3D Data>

Using an MRI device capable of performing MRE measurement, three-dimensional object 3D data 1 of the liver model 1 and three-dimensional object 3D data 2 of the liver model 2 were obtained. The three-dimensional object 3D data were each generated based on model image data obtained by capturing images of the liver model 1 and the liver model 2 with the MRI device and three-dimensional object properties obtained by MRE measurement of the liver model 1 and the liver model 2.

Specifically, the three-dimensional object 3D data each include a plurality of voxels generated based on the model image data, image density information indicating an image density (MRI signal value) in the model image data and allocated to each voxel, and three-dimensional object property information indicating a three-dimensional object property (viscoelasticity) allocated to each voxel.

<Evaluation of Accuracy of Three-Dimensional Object>

### Example 1

Accuracy of the liver model 1 with respect to the liver part was evaluated based on the biological property information included in the obtained medical 3D data of the liver part and the three-dimensional object property information included in the three-dimensional object 3D data 1 of the liver model 1. Specifically, the difference between the three-dimensional object property and the biological property at corresponding parts of the liver part and the liver model 1 was calculated. As a result, the difference was 1%.

### Example 2

Accuracy of the liver model 2 with respect to the liver part was evaluated based on the biological property information included in the obtained medical 3D data of the liver part and the three-dimensional object property information included in the three-dimensional object 3D data 2 of the liver model 2. Specifically, the difference between the three-dimensional object property and the biological property at corresponding parts of the liver part and the liver model 2 was calculated. As a result, the difference was 70%.

In the way described above, the accuracy of the liver model 1 with respect to the liver part was evaluated as being high, and the accuracy of the liver model 2 with respect to the liver part was evaluated as being low.

<Obtainment of Medical 3D Data 2′>

Using an MRI device capable of performing MRE measurement, medical 3D data 2 including liver parts of three healthy persons were obtained. The medical 3D data 2 were generated based on medical image data obtained by capturing images of the healthy persons with the MRI device and biological properties obtained by MRE measurement of the healthy persons. Specifically, each medical 3D data 2 includes a plurality of voxels generated based on the medical image data, image density information indicating an image density (MRI signal value) in the medical image data and allocated to each voxel, and biological property information indicating a biological property (viscoelasticity) allocated to each voxel.

Next, the medical 3D data 2 of the three healthy persons were averaged, to generate medical 3D data 2′ for average healthy persons.

The biological property (viscoelasticity) in the medical 3D data 2′ for average healthy persons was within the ranges of the viscoelasticity of a cured product of the three-dimensional hydrogel object producing composition 1 and the viscoelasticity of a cured product of the three-dimensional hydrogel object producing composition 2 at any local parts.

<Generation of 3D Data 2′ for Object Production>

The medical 3D data 2′ for average healthy persons was subjected to voxel region division, to obtain region-specific medical 3D data 2′ representing the liver part. Next, the region-specific medical 3D data 2′ was converted to a FAV format, to generate FAV-format 3D data 2′ for object production.

<Object Production Example of Liver Model 2′>

The high-strength object producing composition A and the low-strength object producing composition B were loaded into a 3D printer of a material jetting type as illustrated in FIG. 9 capable of expressing strength property gradations, and filled into inkjet heads of the 3D printer. Next, the 3D data 2′ for object production was input into the 3D printer, and a liver model 2′ having a distribution of a strength property corresponding to the distribution of the biological property was produced based on the 3D data 2′ for object production. Specifically, the respective object producing compositions were discharged from the inkjet heads, and formation of a liquid film and curing were repeated sequentially, to produce the model.

<Obtainment of Three-Dimensional Object 3D Data 2′>

Using an MRI device capable of performing MRE measurement, three-dimensional object 3D data 2′ of the liver model 2′ was obtained. The three-dimensional object 3D data 2′ was generated based on model image data obtained by capturing an image of the liver model 2′ with the MRI device and a three-dimensional object property obtained by MRE measurement of the liver model 2′. Specifically, the three-dimensional object 3D data 2′ includes a plurality of voxels generated based on the model image data, image density information indicating an image density (MRI signal value) in the model image data and allocated to each voxel, and three-dimensional object property information indicating a three-dimensional object property (viscoelasticity) allocated to each voxel.

<Evaluation of Accuracy of Liver Model 2′>

Accuracy of the liver model 2′ with respect to the average liver part was evaluated based on the biological property information included in the liver part of the medical 3D data 2′ generated by averaging and the three-dimensional object property information included in the three-dimensional object 3D data 2′ of the liver model 2′. Specifically, the difference between the three-dimensional object property and the biological property at corresponding parts of the average liver part and the liver model 2′ (i.e., difference between an average three-dimensional object property obtained based on ten parts of the liver model 2′ and an average biological property obtained based on corresponding ten parts of the average liver part) was calculated. As a result, the difference was 4%.

<Doctor's Evaluation of Texture of Liver Model 1 and Liver Model 2′>

Five doctors palpated the liver model 1 and the liver model 2′, and compared the results with the texture of actual livers. As a result, the doctors concluded that either liver model had an extremely high reproducibility.

<Production of Training System>

A housing imitating an abdominal shape of a human body having such structures as ribs was produced, and the liver model 1 and structures imitating other organs were disposed inside the housing, to produce a training system 1. A training system 2′ was produced using the liver model 2′ instead of the liver model 1. Holes were opened in part of these housings in order that, for example, laparoscopes or tubes could be inserted. Tools capable of performing an endoscopic surgery were set in the holes.

Next, using biopsy tubes, three doctors performed operations similar to picking a tissue piece from a liver on the liver model 1 and the liver model 2′ in these training systems, to evaluate the texture of the liver model 1 and the liver model 2′. As a result, the doctors concluded that they felt obvious differences between the liver model 1 and the liver model 2′, and that the liver models were close to actual textures.

### REFERENCE SIGNS LIST

10: object producing apparatus
11: three-dimensional hydrogel object producing composition discharging head unit
12: support producing composition discharging head unit
13: ultraviolet irradiator
14: object support substrate
15: stage
16: smoothing member
17: three-dimensional object (hydrogel)
18: support (support material)

1: A three-dimensional object,
wherein the three-dimensional object is produced based on biological property information indicating a biological property obtained by magnetic resonance elastography (MRE) measurement of an organism; and
the three-dimensional object has a distribution of a strength property corresponding to a distribution of the biological property.

2: The three-dimensional object according to claim 1,
wherein the three-dimensional object is produced based on medical 3D data including the biological property information; and
the medical 3D data is generated based on medical image data obtained by capturing an image of the organism with a medical image capturing device and on the biological property.

3: The three-dimensional object according to claim 2,
wherein the medical 3D data includes a plurality of voxels generated based on the medical image data, image density information indicating an image density in the medical image data and allocated to each voxel, and the biological property information allocated to each voxel.

4: The three-dimensional object according to claim 1,
wherein the biological property information is individual biological property information indicating an individual biological property obtained by MRE measurement of one organism.

5: The three-dimensional object according to claim 1,
wherein the biological property information is average biological property information indicating an average biological property obtained by averaging a plurality of individual biological properties obtained by MRE measurement of a plurality of organisms.

6: The three-dimensional object according to claim 1,
wherein a difference of a three-dimensional object property obtained by MRE measurement of the three-dimensional object from the biological property is within 5%.

7: The three-dimensional object according to claim 1,
wherein the three-dimensional object comprises a hydrogel as a material constituting the three-dimensional object.

8: The three-dimensional object according to claim 7,
wherein the hydrogel contains water, a polymer, and a mineral.

9: A training system, comprising:
the three-dimensional object according to claim 1; and
a housing that houses the three-dimensional object.

10: The training system according to claim 9,
wherein the housing is an imitation imitating a structure of at least part of the organism.

11: An object producing method, comprising:
producing a three-dimensional object using a 3D printer based on medical 3D data of an organism,
wherein the medical 3D data includes biological property information indicating a biological property obtained by MRE measurement of the organism; and
the three-dimensional object has a distribution of a strength property corresponding to a distribution of the biological property.

12: The object producing method according to claim 11,
wherein the medical 3D data further includes a plurality of voxels generated based on a medical image data and image density information indicating an image density in the medical image data and allocated to each voxel.

13: The object producing method according to claim 11,
wherein the medical 3D data further includes a plurality of voxels generated based on a medical image data, image density information indicating an image density in the medical image data and allocated to each voxel, and the biological property information indicating the biological property and allocated to each voxel.

14: The object producing method according to claim 1,
wherein the 3D printer is a material jetting type.

15: A method for evaluating accuracy of a three-dimensional object, the method comprising:
evaluating accuracy of property based on biological property information indicating a biological property obtained by MRE measurement of an organism and three-dimensional object property information indicating a property of a three-dimensional object obtained by MRE measurement of the three-dimensional object,
wherein the three-dimensional object is the three-dimensional object according to claim 1.
